(19) United States
(12) Patent Application Publication
(10) Pub. No.: US 2017/0181753 A1
(43) Pub. Date: Jun. 29, 2017

(54) Automatic measuring trauma drill

(71) Applicant: Rolf LANGELAND, Newport, RI (US)

(72) Inventor: Rolf LANGELAND, Newport, RI (US)

(21) Appl. No.: 15/387,896

(22) Filed: Dec. 22, 2016

Related U.S. Application Data
(60) Provisional application No. 62/387,457, filed on Dec. 23, 2015.

(51) Int. Cl. A61B 17/16; A61B 34/00; A61B 90/00

(52) U.S. Cl. CPC A61B 17/1615; A61B 90/06; A61B 34/25; A61B 2090/062; A61B 2090/066; A61B 2017/0003

(57) ABSTRACT

An orthopedic trauma drill is provided that includes an automatic measuring component to ensure that the proper drill depth is achieved for the length of the appropriate screw that is required. The drill may include a stationary sleeve over a drill bit which determines the distance that the drill bit has drilled through a bone, and may provide a real-time numerical readout of the distance measurement. The drill may also include torque sensor to determine the rotation of the drill in revolutions per minute during operation of the drill.

### CROSS-REFERENCE TO RELATED APPLICATIONS

The present application claims the benefit of U.S. Provisional Application No. 62/387,457 filed Dec. 23, 2015, which is hereby incorporated by reference in its entirety.

### BACKGROUND OF THE INVENTION

In medical practice, screws are commonly used in the repair and healing of bone fractures. The process of inserting a screw into a fractured bone typically starts by drilling a hole through the bone that will receive the screw. In certain bones, for example, a drill is passed through the entire bone, including the first cortex, the intramedullary canal and the second cortex. To determine the appropriate length of screw to be inserted, a tool called a depth gauge is then inserted into the drilled hole. Based on the measurement of the depth gauge, a screw having the appropriate length is selected and inserted into the drilled hole.

However, the step of requiring a depth gauge causes the process of inserting a screw to take additional time than may be required. Furthermore, the process of drilling through the bone creates a risk of soft tissue injury around the bone, should the drill extend past the bone.

What is needed therefore is a drill that addresses these problems facing the medical community.

### SUMMARY OF THE INVENTION

The present invention addresses these shortcomings in the art by providing an orthopedic trauma drill that automatically measures the length of a screw to be inserted.

The orthopedic trauma drill according to the present invention provides and meets the current industry standards for orthopedic trauma drills, while also providing many improvements over existing drills.

The orthopedic trauma drill of the present invention provides a significant improvement over the art by providing a drill that comprises an automatic measuring component that ensures that the proper drill depth is achieved for the length of the appropriate screw that is required. The drill according to the present invention will save significant intraoperative time as it eliminates the current need to use a depth gauge in order to determine the appropriate screw length.

In accordance with a first aspect of the invention, an orthopedic drill is provided, including a housing body comprising a motor, a drill bit configured to be powered by the motor for drilling a hole through a bone to receive an orthopedic screw or pin and a sensor configured to measure a distance drilled by the drill bit.

In accordance with an embodiment of the orthopedic drill of the first aspect of the invention, the orthopedic drill further comprises a sleeve positioned over the drill bit. The sleeve may comprise the sensor. During drilling, the sleeve may be configured to remain stationary against the bone as the drill bit moves through the sleeve. The sensor can be configured to measure the distance from the sensor to a tip of the drill bit to determine the distance drilled by the drill bit. In certain instances, the measured distance drilled to penetrate a far cortex of the bone corresponds to a required length of the orthopedic screw or pin.

In accordance with one or more of the above-described embodiments of the orthopedic drill of the first aspect of the invention, the orthopedic drill may further comprise a display screen on the housing body configured to display the measured distance in real-time during drilling.

In accordance with one or more of the above-described embodiments of the orthopedic drill of the first aspect of the invention, the orthopedic drill may further comprise a torque sensor configured to measure the rotation of the drill bit. The rotation of the drill bit changes as the drill bit moves through different layers in the bone having different densities. The required rotation of the drill bit increases during drilling through a first cortex layer of the bone, then decreases during drilling through an intramedullary canal of the bone, then increases during drilling through a second cortex layer of the bone. A measured distance drilled to penetrate the second cortex layer of the bone may correspond to a required length of the orthopedic screw or pin.

In a further embodiment, the orthopedic drill may further comprise a display screen on the housing body configured to display the measured rotation of the drill bit in real-time during drilling. In another further embodiment, the drill bit may be configured to retract when the measured rotation of the drill bit indicates that the drill bit has penetrated a second cortex layer of the bone to prevent damage to tissue around the bone. In a still further embodiment, the orthopedic drill may be configured to provide a visual indicator when the measured rotation of the drill bit indicates that the drill bit has penetrated a second cortex layer of the bone.

In accordance with one or more of the above-described embodiments of the orthopedic drill of the first aspect of the invention, the orthopedic drill may further comprise a detachable electric battery configured to supply electrical power to the motor.

In accordance with one or more of the above-described embodiments of the orthopedic drill of the first aspect of the invention, the orthopedic drill may further comprise at least one lighting element configured to illuminate an area during drilling. The at least one lighting element may be configured to be activated by one or more drill triggers configured to control operation of the drill bit.

### BRIEF DESCRIPTION OF THE FIGURES

FIG. 1 shows a first embodiment of the orthopedic drill according to the present invention.

### DETAILED DESCRIPTION OF THE FIGURES

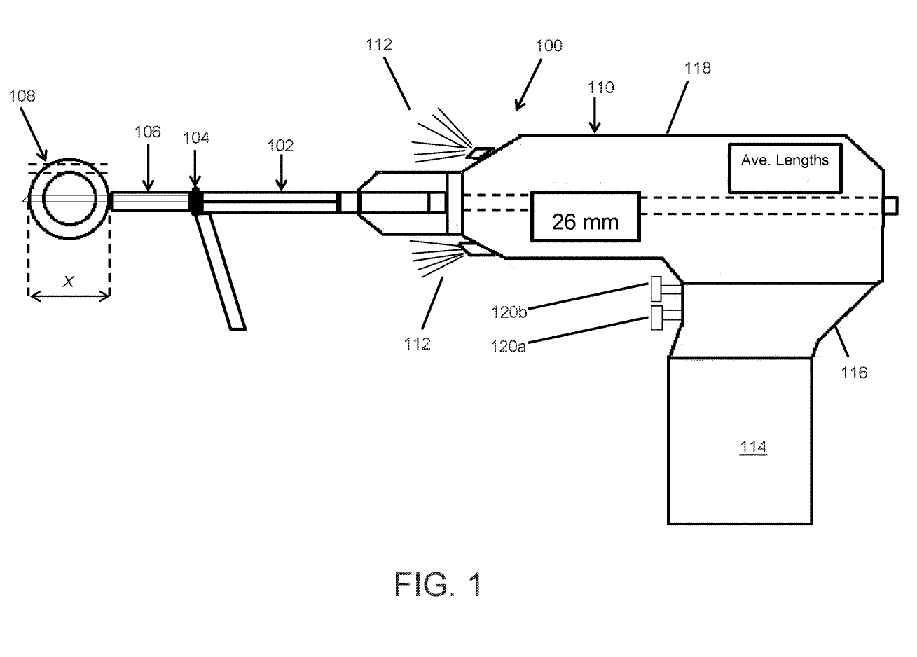

The present invention will now be described with reference made to FIG. 1.

The orthopedic trauma drill 100 of the present invention provides a significant improvement over the art by providing a drill 100 that comprises an automatic measuring component that ensures that the proper drill depth is achieved for the length of the appropriate screw that is required. An example of the trauma drill 100 is shown in FIG. 1. The drill bit 102 is configured to automatically measure the length of the screw.

In accordance with a first embodiment of the invention, the screw length can be measured by using GPS technology. The measurement will gauge off of a stationary sensor 104 of a drill sleeve 106, which can be placed over the drill bit 102, and the drill tip to drill to the appropriate length. During operation of the drill 100, the drill sleeve 106 remains stationary relative to the patient's bone. Thus, while the tip of the drill bit 102 moves through the bone, the drill sleeve 106 remains stationary. The distance from the sensor 104 of the drill sleeve 106 to the moving tip of the drill is measured to determine the distance that the drill tip has travelled. A real-time numerical readout of the measured distance drilled can be provided on a display 110 on the drill 100. The measured distance to the point of penetrating the far second cortex can then be used to determine the appropriate length of screw for insertion.

In a further embodiment of the invention, which may be provided in addition to or alternatively to the above-described measurement system, the screw length can be measured using the drill bit 102 sensation. As previously described, a drill for inserting a medical screw in certain types of bone will pass through a first cortex, an intramedullary canal and a second cortex on the far side of the bone. Because these layers have different compositions and densities, the necessary torque to drill through the layers varies. In accordance with the present invention, a torque or RPM sensor is provided that measures the torque of the drill bit 102. The drill bit torque is increased when drilling into the first cortex, then decreased when in the intramedullary canal, then increased when in the second cortex, and increased again when perforating the second cortex. The change in torque can be measured and utilized to configure the drill 100 to automatically drill to the appropriate depth, as it can determine when the drill bit 102 has reached and perforated the second cortex, and has thus drilled to the necessary depth for inserting a screw.

The torque sensor may also be used in combination with the drill sleeve 106 to provide a measurement of the biocortical bone diameter or screw length. The distance can be determined by measuring from the initial, fixed point of the drill sleeve 106 at the first cortex of the bone and when the change in the RPMs of the drill bit 102 indicates penetration of the second cortex, reading the measurement from the drill sleeve 106.

The drill bit 102 of the trauma drill 100 can be retractable. The torque or RPM sensor on the drill bit 102 can determine the point of the second cortex penetration, and provide signaling to activate retraction of the drill bit 102 to prevent damage to vascular structures and soft tissues around the bone. Transcortical drilling can be recognized by the torque or the RPM of the drill bit 102 not changing, and a warning 108 can be provided, for example, in the form of a visual indicator on the display 110 or lights 112 having a particular color.

The drill 100 according to the invention further includes a display 110, such as an LED display to indicate the depth of the drilling or screw length in real-time as the drilling occurs. The total depth (x) at the point of the second cortex penetration, can be presented on the display 110.

The drill 100 may further include one or more lights 112. The lights 112 can include a neon light, LED or other suitable source of light, which is configured to automatically switch on when the drill is on. This allows for illuminating the working area when the drill 100 is in operation.

The drill 100 may be configured with a detachable battery clip 114 for a cordless power configuration. The battery clip 114 may be attached to or form part of the grip 116 of the drill 100. In alternative embodiments, the drill 100 can be provided with a connector for connecting the drill 100 to an alternative power source, such as an electrical outlet.

The drill 100 may include a motor or other device for generating the rotating motion in the drill bit 102, as would be known in the art of orthopedic drills. The motor may be contained within the housing body 118 of the drill 100. The housing body 118 may also include the LED display 110 on its outer surface, and in certain embodiments, may include an identification of the average diameters of certain bones that may be drilled frequently. One or more actuators can be provided on the drill 100 for controlling operation of the drill bit 102 and motor. For example, as shown in FIG. 1, a forward drill trigger 120a and reverse drill trigger 120b can be provided for operating the drill 100.

The drill triggers 120a and/or 120b may be also be configured to turn on the lights 112 of the drill 100 when actuated, and turn off the lights 112 when released.

The drill according to the invention may be configured for use and provided with various standard drill attachments, including but not limited to an AO drill bit attachment, a pin driver attachment, a sag saw attachment, and a hand tightening chuck attachment for tightening bits inserted into a chuck of the drill 100.

According to a further embodiment of the trauma drill of the invention, a Gatling-gun type screw driver is provided. This embodiment of the trauma drill includes a Gatling-type spin chamber, within which the screws are contained. This second gun system can be provided in wireless communication with the auto-measuring trauma drill 100. The screw-driver automatically dials in the appropriate screw length and is ready for power screw insertion with a torque limited driver being provided to avoid stripping of screw. The screw driver can also be manually set to the desired screw length.

The screw driver can be configured for various sized screws depending on the bone that it is required for, including for example, 4.5 mm for large bone (e.g., tibia, femur), 3.5 mm for medium bone (e.g., humerus) and 2.7 mm for small bone (e.g., fibula, radius, ulna).

While there have been shown and described and pointed out fundamental novel features of the invention as applied to preferred embodiments thereof, it will be understood that various omissions and substitutions and changes in the form and details of the devices and methods described may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice.

What is claimed:

1. An orthopedic drill comprising:
a housing body comprising a motor,
a drill bit configured to be powered by the motor for drilling a hole through a bone to receive an orthopedic screw or pin; and
a sensor configured to measure a distance drilled by the drill bit.

2. The orthopedic drill according to claim 1, further comprising a sleeve positioned over the drill bit.

3. The orthopedic drill according to claim 2, wherein the sleeve comprises the sensor.

4. The orthopedic drill according to claim 3, wherein the sleeve is configured to remain stationary against the bone and the drill bit moves through the sleeve during drilling.

5. The orthopedic drill according to claim 4, wherein the sensor is configured to measure the distance from the sensor to a tip of the drill bit to determine the distance drilled by the drill bit.

6. The orthopedic drill according to claim 5, wherein a measured distance drilled to penetrate a far cortex of the bone corresponds to a required length of the orthopedic screw or pin.

7. The orthopedic drill according to claim 1, further comprising a display screen on the housing body configured to display the measured distance in real-time during drilling.

8. The orthopedic drill according to claim 5, further comprising a display screen on the housing body configured to display the measured distance in real-time during drilling.

9. The orthopedic drill according to claim 1, further comprising a torque sensor configured to measure the rotation of the drill bit.

10. The orthopedic drill according to claim 9, wherein the rotation of the drill bit changes as the drill bit moves through different layers in the bone having different densities.

11. The orthopedic drill according to claim 10, wherein a required rotation of the drill bit increases during drilling through a first cortex layer of the bone, then decreases during drilling through an intramedullary canal of the bone, then increases during drilling through a second cortex layer of the bone.

12. The orthopedic drill according to claim 10, further comprising a display screen on the housing body configured to display the measured rotation of the drill bit in real-time during drilling.

13. The orthopedic drill bit according to claim 10, wherein the drill bit is configured to retract when the measured rotation of the drill bit indicates that the drill bit has penetrated a second cortex layer of the bone to prevent damage to tissue around the bone.

14. The orthopedic drill bit according to claim 10, wherein the orthopedic drill is configured to provide a visual indicator when the measured rotation of the drill bit indicates that the drill bit has penetrated a second cortex layer of the bone.

15. The orthopedic drill according to claim 5, further comprising a torque sensor configured to measure the rotation of the drill bit.

16. The orthopedic drill according to claim 15, wherein the rotation of the drill bit changes as the drill bit moves through different layers in the bone having different densities.

17. The orthopedic drill according to claim 16, wherein the torque sensor is configured to determine when the drill bit has penetrated a second cortex layer of the bone based on changes in the rotation of the drill bit, and wherein a measured distance drilled to penetrate the second cortex layer of the bone corresponds to a required length of the orthopedic screw or pin.

18. The orthopedic drill according to claim 15, further comprising a display screen on the housing body configured to display the measured rotation of the drill bit the measured distance drilled in real-time during drilling.

19. The orthopedic drill according to claim 1, further comprising a detachable electric battery configured to supply electrical power to the motor.

20. The orthopedic drill according to claim 19, further comprising at least one lighting element configured to illuminate an area during drilling, wherein the at least one lighting element is configured to be activated by one or more drill triggers configured to control operation of the drill bit.
